(19) United States
(12) Patent Application Publication
(10) Pub. No.: US 2021/0321905 A1
(43) Pub. Date: Oct. 21, 2021

(54) APPARATUS AND METHOD FOR MEASURING GROUND REACTION FORCE

(71) Applicant: ELECTRONICS AND TELECOMMUNICATIONS RESEARCH INSTITUTE, Daejeon (KR)

(72) Inventors: Yong Wan KIM, Daejeon (KR); Daehwan KIM, Daejeon (KR); Ki Suk LEE, Daejeon (KR)

(21) Appl. No.: 17/234,466

(22) Filed: Apr. 19, 2021

(30) Foreign Application Priority Data
Apr. 21, 2020 (KR) 10-2020-0047911

(51) Int. Cl. A61B 5/103

(52) U.S. Cl. CPC A61B 5/1038; A61B 2562/0247; A61B 2562/0219

(57) ABSTRACT

An apparatus and method for measuring a ground reaction force may be disclosed. The apparatus may include a ground reaction force measurement unit configured to calculate a first ground reaction force value using a human body proportional mass parameter value for a subject to be measured and inertial data measured for the subject, a foot pressure measurement unit configured to sense a foot pressure for the subject and calculate foot pressure data using the foot pressure, and a final ground reaction force calculation unit configured to calculate a second ground reaction force value using the foot pressure data and the first ground reaction force value.

### CROSS-REFERENCE TO RELATED APPLICATION

This application claims priority to and the benefit of Korean Patent Application No. 10-2020-0047911 filed in the Korean Intellectual Property Office on Apr. 21, 2020, the entire contents of which are incorporated herein by reference.

### BACKGROUND OF THE INVENTION

### (a) Field of the Invention

The present invention relates to an apparatus and method for measuring a ground reaction force.

### (b) Description of the Related Art

Recently, due to aging, the number of elderly people complaining of gait abnormalities due to nerve and muscle damage is increasing. Accordingly, there is a need for a method for collecting biological signal data that can easily diagnose and predict musculoskeletal diseases.

Particularly, gait motion is standardized as the most basic motion for musculoskeletal diagnosis and has a general pattern. Accordingly, gait analysis using biomechanical information extracted from each joint is widely used for musculoskeletal diagnosis and prescription. The joint force and joint moment estimated in the lower limb are generally used as important factors in evaluating patients in clinical and rehabilitation fields, and diagnosis can be performed using minute differences in joint moments.

In general, it is essential to measure a ground reaction force using a force plate for gait analysis among biological signals. However, it is not possible to measure continuous steps on the force plate, and a plurality of force plates must be installed according to the stride length, and the cost burden is large due to expensive equipment.

The above information disclosed in this Background section is only for enhancement of understanding of the background of the invention, and therefore it may contain information that does not form the prior art that is already known in this country to a person of ordinary skill in the art.

### SUMMARY OF THE INVENTION

The present invention is to provide a method and apparatus for simply measuring a ground reaction force.

In one aspect, an apparatus for measuring a ground reaction force may be provided. The apparatus may include a ground reaction force measurement unit configured to calculate a first ground reaction force value using a human body proportional mass parameter value for a subject to be measured and inertial data measured for the subject, a foot pressure measurement unit configured to sense a foot pressure for the subject and calculate foot pressure data using the foot pressure, and a final ground reaction force calculation unit configured to calculate a second ground reaction force value using the foot pressure data and the first ground reaction force value.

The ground reaction force measurement unit may include an inertial sensor configured to be attached to the subject and sense the inertial data, and a ground reaction force calculator configured to calculate the first ground reaction force value by performing an inverse kinematic analysis using the inertial data and the human body proportional mass parameter value.

The ground reaction force measurement unit may further include a vision sensor configured to sense vision data for the subject, and the ground reaction force calculator may calculate the first ground reaction force value using the inertial data, the human body proportional mass parameter value, and the vision data.

The inertial data may include linear acceleration and rotation acceleration.

The apparatus may further include a human body proportional mass parameter calculation unit configured to calculate the human body proportional mass parameter value using a measurement prediction standard model for a human body mass distribution.

The foot pressure measurement unit may include a foot pressure sensor configured to sense the foot pressure, and a foot pressure data calculator configured to calculate the foot pressure data including a foot pressure distribution and a foot pressure center using the foot pressure.

The foot pressure data calculator may calculate a walking period for the subject using the foot pressure distribution.

The final ground reaction force calculation unit may calculate a weight movement ratio between both feet of the subject using the foot pressure distribution or the foot pressure center, and reflects the weight movement ratio to the first ground reaction force value to calculate the second ground reaction force value.

The final ground reaction force calculation unit may delete a portion of the first ground reaction force value for a foot that is not in contact with a ground by using the walking period.

The apparatus may further include a matching unit configured to match coordinates between the first ground reaction force value and the foot pressure data.

The apparatus may further include a ground reaction force correction unit configured to include: a peak value estimator for estimating a peak value of the ground reaction force by using the coordinate-matched first ground reaction force value, the coordinate-matched foot pressure data, and the human body proportional mass parameter value; and a corrector for correcting the first ground force value by using the peak value.

The peak value estimator may estimate the peak value by performing neural network learning using the coordinate-matched first ground reaction force value, the coordinate-matched foot pressure data, and the human body proportional mass parameter value.

In another aspect, a method of measuring a ground reaction force against a subject to be measured by a ground reaction force measuring apparatus may be provided. The method may include measuring inertial data for the subject, calculating a first ground reaction force value by using a human body proportional mass parameter value for the subject and the inertial data, sensing a foot pressure for the subject, calculating foot pressure data by using the foot pressure, and calculating a second ground reaction force value by using the foot pressure data and the first ground reaction force value.

The method may further include sensing vision data for the subject, and the calculating the first ground reaction force value may include calculating the first ground reaction force value by using the inertial data, the human body proportional mass parameter value, and the vision data.

The inertial data may include linear acceleration and rotation acceleration.

The foot pressure data may include foot pressure distribution and a foot pressure center.

The calculating the foot pressure data may include calculating a walking period for the subject using the foot pressure distribution, and the calculating a second ground reaction force value may include deleting a portion of the first ground reaction force value for a foot that is not in contact with a ground by using the walking period.

The calculating the second ground reaction force value may include: calculating a weight movement ratio between both feet of the subject by using the foot pressure distribution or the foot pressure center; and calculating the second ground reaction force value by reflecting the weight movement ratio to the first ground reaction force value.

In another aspect, a method of measuring a ground reaction force against a subject to be measured by a ground reaction force measuring apparatus may be provided. The method may include measuring inertial data for the subject, calculating a first ground reaction force value by using a human body proportional mass parameter value for the subject and the inertial data, sensing a foot pressure for the subject, calculating a walking period for the subject by using the foot pressure, and calculating a final ground reaction force value by using the walking period and the first ground reaction force value.

The method may further include sensing vision data for the subject, and the calculating the first ground reaction force value may include calculating the first ground reaction force value by using the inertial data, the human body proportional mass parameter value, and the vision data.

According to one embodiment, by using inertia data and foot pressure data, it is possible to simply calculate a ground reaction force value.

According to one embodiment, by using inertia data and foot pressure data, it is possible to calculate a ground reaction force value at a low cost.

### BRIEF DESCRIPTION OF THE DRAWINGS

FIG. 1 is a block diagram showing a ground reaction force measuring apparatus according to one embodiment.

FIG. 2 is a block diagram showing a ground reaction force measuring unit according to one embodiment.

FIG. 3 is a block diagram showing a foot pressure measurement unit according to one embodiment.

FIG. 4A is a diagram showing a pressure value and a foot pressure center according to one embodiment, and FIG. 4B is a diagram showing a foot pressure distribution according to one embodiment.

FIG. 5 is a diagram conceptually showing a walking period according to one embodiment.

FIG. 6 is a block diagram showing a ground reaction force correction unit 150 according to one embodiment.

FIG. 7 is a diagram showing a first ground reaction force value according to one embodiment.

FIG. 8 is a block diagram showing a final ground reaction force calculation unit 160 according to one embodiment.

FIG. 9 is a diagram showing a computer system according to one embodiment.

### DETAILED DESCRIPTION OF THE EMBODIMENTS

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Throughout the specification, when an element, such as a layer, region, or substrate, is described as being “on,” “connected to,” or “coupled to” another element, it may be directly “on,” “connected to,” or “coupled to” the other element, or there may be one or more other elements intervening therebetween. In contrast, when an element is described as being “directly on,” “directly connected to,” or “directly coupled to” another element, there can be no other elements intervening therebetween.

As used herein, the term “and/or” includes any one and any combination of any two or more of the associated listed items.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in examples described herein may also be referred to as a second member, component, region, layer, or section without departing from the teachings of the examples.

Spatially relative terms such as “above,” “upper,” “below,” and “lower” may be used herein for ease of description to describe one element's relationship to another element as shown in the figures. Such spatially relative terms are intended to encompass different orientations of the device in use or operation in addition to the orientation depicted in the figures. For example, if the device in the figures is turned over, an element described as being “above” or “upper” relative to another element will then be “below” or “lower” relative to the other element. Thus, the term “above” encompasses both the above and below orientations depending on the spatial orientation of the device. The device may also be oriented in other ways (for example, rotated 90 degrees or at other orientations), and the spatially relative terms used herein are to be interpreted accordingly.

The terminology used herein is for describing various examples only, and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

The features of the examples described herein may be combined in various ways as will be apparent after an understanding of the disclosure of this application. Further, although the examples described herein have a variety of configurations, other configurations are possible as will be apparent after an understanding of the disclosure of this application.

In addition, unless explicitly described to the contrary, the word “comprise” and variations such as “comprises” or “comprising” will be understood to imply the inclusion of stated elements but not the exclusion of any other elements.

An apparatus and method for measuring a ground reaction force according to one embodiment measures a ground reaction force value, which is an essential element in gait analysis. An apparatus and method for measuring a ground reaction force according to one embodiment will be described in detail below.

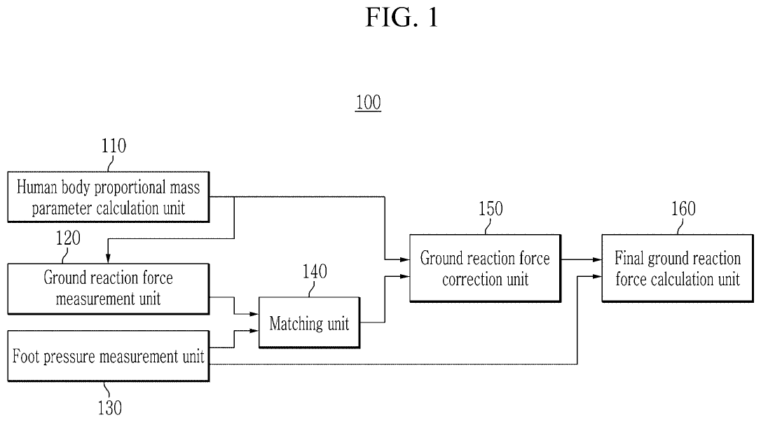

FIG. 1 is a block diagram showing a ground reaction force measuring apparatus 100 according to one embodiment.

As shown in FIG. 1, the ground reaction force measuring apparatus 100 includes a human body proportional mass parameter calculation unit 110, a ground reaction force measurement unit 120, a foot pressure measurement unit 130, a matching unit 140, a ground reaction force correction unit 150, and a final ground reaction force calculation unit 160.

The human body proportional mass parameter calculation unit 110 calculates a human body proportional mass parameter value for an object for which the ground reaction force is to be measured. Here, the object for which the ground reaction force is to be measured may be a person. Hereinafter, the term “subject” is used for the object for which the ground reaction force is to be measured.

The ground reaction force measurement unit 120 calculates a first ground reaction force value through inverse kinematic analysis using the human body proportional mass parameter value calculated by the human body proportional mass parameter calculation unit 110 and sensing data measured using an inertial sensor. Meanwhile, the ground reaction force measurement unit 120 may improve the accuracy of the ground reaction force calculation by using sensing data acquired through the vision sensor as an aid when calculating the first ground reaction force value.

The foot pressure measurement unit 130 calculates foot pressure data using sensing data measured through a foot pressure sensor. Here, the foot pressure measurement unit 130 may calculate a foot pressure center, a foot pressure distribution, and a walking period as foot pressure data.

The matching unit 140 matches coordinates between the first ground reaction force value calculated by the ground reaction force measurement unit 120 and the foot pressure data calculated by the foot pressure measurement unit 130.

The ground reaction force correction unit 150 estimates the peak value of the ground reaction force by performing neural network learning on the human body proportional mass data calculated by the human body proportional mass parameter calculation unit 110, the first ground reaction force value, and the foot pressure data matched by the matching unit 140. Further, the ground reaction force correction unit 150 corrects the first ground reaction force value by using the estimated peak value.

The final ground reaction force calculation unit 160 finally calculates a ground reaction force value using the foot pressure data calculated by the foot pressure measurement unit 130 and the first ground reaction force value corrected by the ground reaction force correction unit 150.

Each component of such a ground reaction force measuring apparatus 100 will be described in detail below.

The human body proportional mass parameter calculation unit 110 calculates a human body proportional mass parameter value of a subject using a measurement prediction standard model for a human body mass distribution. Here, the measurement prediction standard model for the human body mass distribution may be a Zatsiorsky-Seluyanov model. The Zatsiorsky-Seluyanov model presents data such as length of each joint, center of mass of each joint, and mass distribution of each joint corresponding to data of human body information (body weight, height, etc.). The human body proportional mass parameter calculation unit 110 according to one embodiment inputs the subject's body information (e.g., height, weight, length of a joint, etc.) into the measurement prediction standard model for the human body mass distribution to calculate the human body proportional mass data of the subject. Here, the human body proportional mass data may include a length of each joint, a center of mass of each joint, and a mass distribution of each joint.

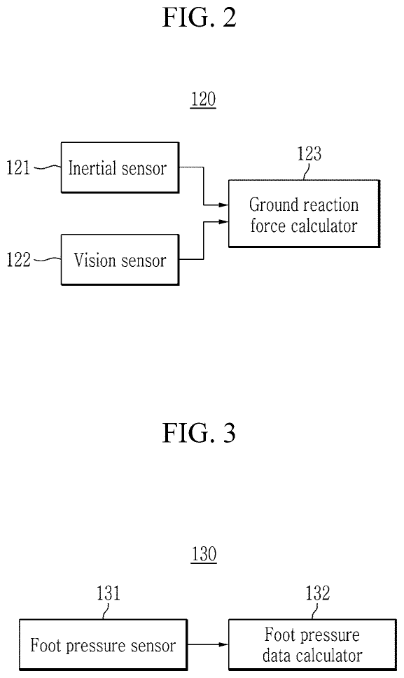

FIG. 2 is a block diagram showing a ground reaction force measuring unit 120 according to one embodiment.

As shown in FIG. 2, the ground reaction force measurement unit 120 according to one embodiment includes an inertial sensor 121, a vision sensor 122, and a ground reaction force calculator 123.

The inertial sensor 121 is attached to the appropriate position of each lower limb joint of the subject and senses inertial data. Here, the inertial sensor 121 may be attached to each lower limb joint of the subject based on the human body proportional mass data of the subject calculated by the human body proportional mass parameter calculation unit 110.

The inertial sensor 121 senses (measures) linear acceleration and rotation acceleration of each joint when the subject is walking. The inertial sensor 121 can be implemented through an acceleration sensor and a gyro sensor. The method for sensing linear acceleration and rotation acceleration through the inertial sensor 121 can be known by a person of ordinary skill in the technical field to which the present invention belongs, and thus a detailed description thereof will be omitted. The linear acceleration and rotation acceleration that are sensed reflect the specific force of each joint and the angular ratio of each joint.

The vision sensor 122 senses the vision data of the subject while the subject is walking. The vision sensor 122 can be implemented as a depth camera, and the subject takes a rest position in front of the vision camera and then walks. At this time, the vision sensor 122 senses the vision data of the subject. The method of sensing vision data through the vision sensor 122 can be known to a person of ordinary skill in the technical field to which the present invention belongs, and thus a detailed description thereof will be omitted.

The ground reaction force calculator 123 calculates the acceleration at the joint's center of mass, the force at the part where the joints are connected, the external force acting on the joint, and the gravitational acceleration by using the linear acceleration and rotation acceleration of each joint sensed by the inertial sensor 121. Then, the ground reaction force calculator 123 calculates the first ground reaction force acting on the lower limb joint by using the calculated value and the human body proportional mass parameter value calculated by the human body proportional mass parameter calculation unit 110.

Here, the ground reaction force calculation unit 123 calculates a reaction force acting on each joint by repeatedly performing inverse kinematic analysis from the uppermost joint to the lowermost joint. Through this, the first ground reaction force value is calculated. The method for calculating the ground reaction force value through the inverse kinematics analysis can be seen by a person of ordinary skill in the technical field to which the present invention belongs, and a detailed description thereof will be omitted.

Meanwhile, the ground reaction force calculator 123 can increase the accuracy of the ground reaction force calculation by additionally using the vision data sensed by the vision sensor 122 when calculating the first ground reaction force value. The position of each joint to which the inertial sensor 121 is attached has a limitation in expressing the exact position of the joint. Accordingly, the ground reaction force calculator 123 may additionally use the vision data to calculate the first ground reaction force value by estimating the position (pose) of each joint through the vision data acquired by the vision sensor 122. Meanwhile, the ground reaction force calculator 123 estimates the pose of the joint by applying extended Kalman filtering to the vision data acquired by the vision sensor 122.

Meanwhile, the ground reaction force calculator 123 can remove high-frequency noise by performing low-pass filtering suitable for the movement frequency of the human body on the data sensed by the inertial sensor 121 and the data sensed by the vision sensor 122. This prevents amplification of the error for the first ground reaction force value calculated by the ground reaction force calculator 123.

As such, the first ground reaction force value calculated by the ground reaction force calculator 123 includes the vertical ground reaction force value, the horizontal (lateral) ground reaction force value, and the anterior ground reaction force value for each of the left and right feet.

The first ground reaction force value calculated by the ground reaction force measurement unit 120 is a value to which segmentation of data according to the walking period is not applied. That is, the first ground reaction force value includes the calculated ground reaction force value even for the feet of the subject who is not in contact with the ground. Accordingly, the ground reaction force measuring apparatus 100 according to one embodiment performs foot pressure sensing by the foot pressure measurement unit 130 in order to improve the accuracy of the ground reaction force and quickly reflect the ground reaction force value according to the walking period.

FIG. 3 is a block diagram showing a foot pressure measurement unit 130 according to one embodiment.

As shown in FIG. 3, the foot pressure measurement unit 130 according to one embodiment includes a foot pressure sensor 131, and a foot pressure data calculator 132.

The foot pressure sensor 131 senses the foot pressure of a subject performing a walking motion on the foot pressure sensor 131. The foot pressure sensor 131 may be a sensor in which a Force Sensitive Resistor (FSR) sensor is formed in a lattice structure of upper, lower, left, and right. The subject performs a walking motion on an FSR sensor composed of a lattice structure. The FSR sensor is a sensor that uses the property that the resistance value changes according to the load such as physical force and weight, and has a low cost and simple structure.

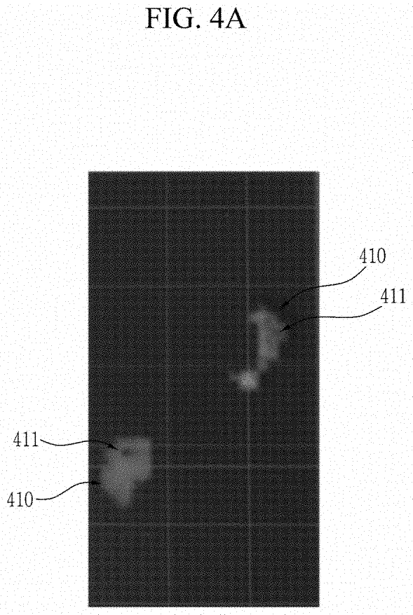

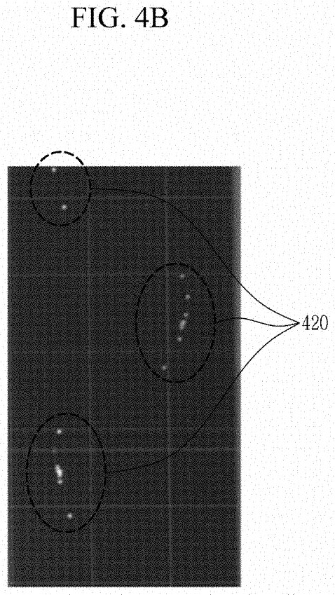

Foot pressure data calculator 132 calculates foot pressure data using the pressure value sensed by foot pressure sensor 131. Since the pressure value sensed by the foot pressure sensor 131 is not a spatially continuous value, the foot pressure data calculator 132 calculates the foot pressure distribution by 2D interpolating the sensing points. The foot pressure data calculator 132 calculates the center of pressure for each frame using the pressure value sensed by the foot pressure sensor 131. Here, the resultant force of the foot pressure distribution acts through a foot pressure center P (a point on the 2D plane). The distribution of the foot pressure center calculated every frame corresponds to the foot pressure distribution. FIG. 4A is a diagram showing a pressure value and a foot pressure center according to one embodiment, and FIG. 4B is a diagram showing a foot pressure distribution according to one embodiment. In FIG. 4A, 410 denotes the pressure value sensed by the foot pressure sensor 131, and 411 denotes the foot pressure center. In FIG. 4B, 420 represents the foot pressure distribution. Meanwhile, connecting the foot pressure center of each frame is the center of pressure trajectory (i.e., foot pressure distribution) against the ground reaction force applied to the foot during walking. Through this center of pressure trajectory, the action point of the ground reaction force can be known.

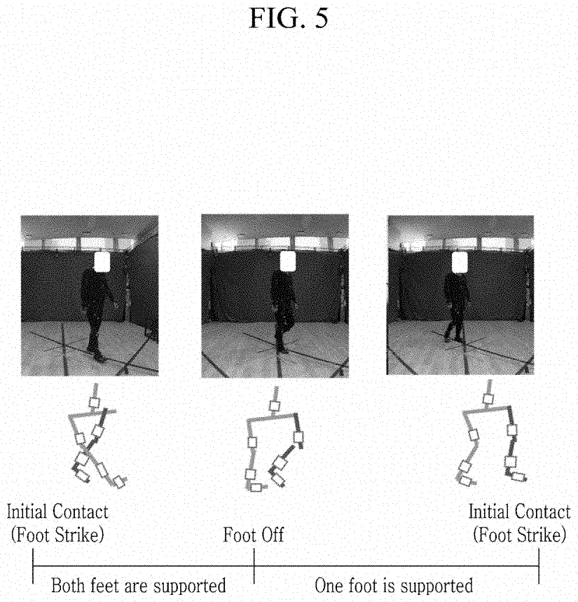

The foot pressure data calculator 132 can calculate biomechanical parameters such as stride length, stride width, foot angle, heel contact time, toe contact time, stride time, swing time, and stance time by using the calculated foot pressure distribution. Particularly, the foot pressure data calculator 132 according to one embodiment can calculate the walking cycle corresponding to an initial contact (foot strike) of the left foot, a foot off, and an initial contact of the right foot by using the foot pressure distribution. FIG. 5 is a diagram conceptually showing a walking period according to one embodiment. As shown in FIG. 5, both feet are supported between an initial contact (foot strike) of the left foot and the foot off, and one foot is supported between the foot off and an initial contact (foot strike) of the right foot. In the above, the first ground reaction force value calculated by the ground reaction force measurement unit 120 also includes a ground reaction force value for the unsupported foot (foot floating in the air) in the one foot support state. Accordingly, the final ground reaction force calculation unit 160 described below performs segmentation on the first ground reaction force value by deleting the ground reaction force value for the foot floating in the air in the state of supporting one foot using the walking period.

The matching unit 140 matches coordinates between the first ground reaction force value calculated by the ground reaction force measurement unit 120 and the foot pressure data calculated by the foot pressure measurement unit 130. Since the first ground reaction force value calculated by the ground reaction force measurement unit 120 is based on the position of the joint, it is a three-dimensional coordinate system, and the foot pressure data measured by the foot pressure measurement unit 130 is a two-dimensional coordinate system. Accordingly, the matching unit 140 matches where the foot pressure data corresponds to the data in the 3D coordinate system. In addition, the matching unit 140 may adjust the normalization and data offset for the two sets of data when the coordinates are matched for two sets of data.

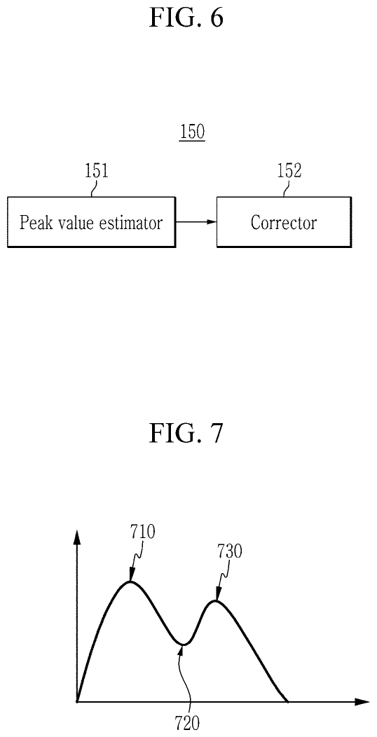

FIG. 6 is a block diagram showing a ground reaction force correction unit 150 according to one embodiment.

As shown in FIG. 6, the ground reaction force correction unit 150 according to one embodiment includes a peak value estimator 151 and a corrector 152.

The peak value estimator 151 estimates a peak value of the ground reaction force by performing neural network learning on the human body proportional mass data calculated by the human body proportional mass parameter calculation unit 110, the first ground reaction force value, and foot pressure data matched by the matching unit 140. Here, for neural network learning, a multi-layer perceptron neural network may be used. First, the peak value estimator 151 trains a multi-layer perceptron neural network through the reference measured value measured by a force plate, the human body proportional mass data calculated by the human body proportional mass parameter calculation unit 110, and the foot pressure data. The peak value estimator 151 estimates the peak value of the ground reaction force by inputting the human body proportional mass data calculated by the human body proportional mass parameter calculation unit 110, the first ground reaction force value, and foot pressure data matched by the matching unit 140 as inputs of the trained multi-layer perceptron neural network. The ground reaction force value measured on the existing force plate may be more accurate than the first ground reaction force value according to the embodiment. Accordingly, a process of estimating a peak value for the first ground reaction force value after training the multi-layer perceptron neural network through the reference measured value measured by the force plate is performed.

FIG. 7 is a diagram showing a first ground reaction force value according to one embodiment. As shown in FIG. 7, the first ground reaction force value may have three peak values 710, 720, and 730. The peak value estimator 151 according to one embodiment estimates the ground reaction force peak value through the multi-layer perceptron neural network so that the three peak values of FIG. 7 are similar to the ground reaction force peak value of the force plate.

The corrector 152 corrects the entire first ground reaction force value (the first ground reaction force value calculated by the ground reaction force measurement unit 120 or the first ground reaction force value matched by the matching unit 140) by using the peak value estimated by the peak value estimator 151. That is, the corrector 152 entirely corrects the first ground reaction force value according to the estimated peak value.

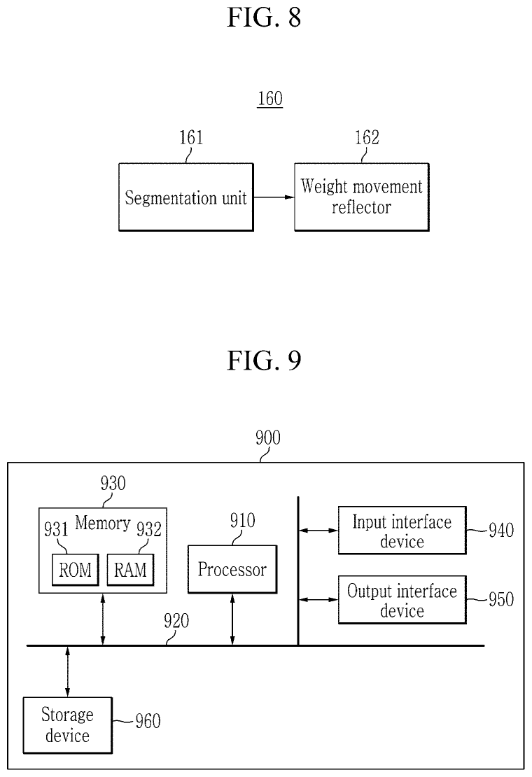

FIG. 8 is a block diagram showing a final ground reaction force calculation unit 160 according to one embodiment.

As shown in FIG. 8, the final ground reaction force calculation unit 160 according to one embodiment includes a segmentation unit 161 and a weight movement reflector 162.

The segmentation unit 161 performs data segmentation on the first ground reaction force value corrected by the ground reaction force correction unit 150 using the walking period measured by the foot pressure measurement unit 130. As described above, the first ground reaction force value and the first ground reaction force value with a corrected peak value includes a ground reaction force value measured even for the foot of the subject that is not in contact with the ground. Accordingly, the segmentation unit 161 determines the first ground reaction force value corresponding to the foot of the subject that is not in contact with the ground using the walking period measured by the foot pressure measurement unit 130, and deletes the first ground reaction force value of the corresponding part.

In addition, the weight movement reflector 162 calculates the weight movement ratio between the left and right feet using the foot pressure data calculated by the foot pressure measurement unit 130, and corrects the first ground reaction force value based on the calculated weight movement ratio. The first ground reaction force value (i.e., the first ground reaction force value calculated by the ground reaction force measurement unit 120, the first ground reaction force value corrected by the ground reaction force correction unit 150, and the first ground reaction force value corrected by the segmentation unit 161) is a ground reaction force value assuming support of one foot in which both feet are not in contact with the ground. When calculating the ground reaction force value for the left foot by inverse kinematic analysis, the entire upper body is also used in calculation, and when calculating the ground reaction force on the right foot by inverse kinematic analysis, the entire upper body is also used in calculation. Accordingly, since the upper body part is reflected redundantly, it is necessary to reflect the weight movement between both feet when walking.

The weight movement reflecting unit 162 according to one embodiment may calculate a weight movement ratio between both feet through two methods. As a first method, the weight movement reflector 162 may calculate a weight movement ratio between both feet using the foot pressure center calculated by the foot pressure measuring unit 130. That is, the weight movement reflector 162 may calculate the weight movement ratio by calculating the pressure value at the center of the foot pressure of the left foot and the pressure value at the center of the foot pressure of the right foot as a ratio. As a second method, the weight movement reflector 162 may calculate a weight movement ratio between both feet using the foot pressure distribution calculated by the foot pressure measuring unit 130. That is, the weight movement reflector 162 calculates the average pressure distribution value of the left foot using the pressure distribution of the left foot, and calculates the average pressure distribution value of the right foot using the pressure distribution of the right foot. The weight movement reflector 162 may set a ratio of the calculated two average pressure distribution values as the weight movement ratio.

The weight movement reflector 162 finally calculates a ground reaction force value by reflecting the calculated weight movement ratio to the first ground reaction force value.

As described above, the ground reaction force measuring apparatus 100 according to one embodiment may calculate a ground reaction force value using a relatively inexpensive inertial sensor and a foot pressure sensor even without using an expensive force plate device. The ground reaction force value calculated as described above can be used when diagnosing a musculoskeletal patient.

FIG. 9 is a diagram showing a computer system 900 according to one embodiment.

The ground reaction force measuring apparatus 100 according to one embodiment may be implemented in the computer system 900 of FIG. 9. Each component of the ground reaction force measuring apparatus 100 can also be implemented in the computer system 900 of FIG. 9.

The computer system 900 can include at least one of a processor 910, a memory 930, an input interface device 940, an output interface device 950, and a storage device 960, that communicate via a bus 920.

The processor 910 can be a central processing (CPU) or a semiconductor device that executes instructions stored in the memory 930 or the storage device 960. The processor 910 can be configured to implement the functions and methods described in FIG. 1 to FIG. 8.

The memory 930 and the storage device 960 can include various forms of volatile or non-volatile storage media. For example, the memory 930 can include a read only memory (ROM) 931 or a random access memory (RAM) 932. In one embodiment, the memory 930 may be located inside or outside the processor 910, and the memory 930 can be coupled to the processor 910 through various already-known means.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An apparatus for measuring a ground reaction force, the apparatus comprising:
a ground reaction force measurement unit configured to calculate a first ground reaction force value using a human body proportional mass parameter value for a subject to be measured and inertial data measured for the subject;
a foot pressure measurement unit configured to sense a foot pressure for the subject and calculate foot pressure data using the foot pressure; and
a final ground reaction force calculation unit configured to calculate a second ground reaction force value using the foot pressure data and the first ground reaction force value.

2. The apparatus of claim 1, wherein
the ground reaction force measurement unit includes:
an inertial sensor configured to be attached to the subject and sense the inertial data; and
a ground reaction force calculator configured to calculate the first ground reaction force value by performing an inverse kinematic analysis using the inertial data and the human body proportional mass parameter value.

3. The apparatus of claim 2, wherein
the ground reaction force measurement unit further includes a vision sensor configured to sense vision data for the subject, and
the ground reaction force calculator calculates the first ground reaction force value using the inertial data, the human body proportional mass parameter value, and the vision data.

4. The apparatus of claim 2, wherein
the inertial data includes linear acceleration and rotation acceleration.

5. The apparatus of claim 1, further comprising
a human body proportional mass parameter calculation unit configured to calculate the human body proportional mass parameter value using a measurement prediction standard model for a human body mass distribution.

6. The apparatus of claim 1, wherein
the foot pressure measurement unit includes:
a foot pressure sensor configured to sense the foot pressure; and
a foot pressure data calculator configured to calculate the foot pressure data including a foot pressure distribution and a foot pressure center using the foot pressure.

7. The apparatus of claim 6, wherein
the foot pressure data calculator calculates a walking period for the subject using the foot pressure distribution.

8. The apparatus of claim 6, wherein
the final ground reaction force calculation unit calculates a weight movement ratio between both feet of the subject using the foot pressure distribution or the foot pressure center, and reflects the weight movement ratio to the first ground reaction force value to calculate the second ground reaction force value.

9. The apparatus of claim 7, wherein
the final ground reaction force calculation unit deletes a portion of the first ground reaction force value for a foot that is not in contact with a ground by using the walking period.

10. The apparatus of claim 1, further comprising
a matching unit configured to match coordinates between the first ground reaction force value and the foot pressure data.

11. The apparatus of claim 10, further comprising
a ground reaction force correction unit configured to include: a peak value estimator for estimating a peak value of the ground reaction force by using the coordinate-matched first ground reaction force value, the coordinate-matched foot pressure data, and the human body proportional mass parameter value; and a corrector for correcting the first ground force value by using the peak value.

12. The apparatus of claim 11, wherein
the peak value estimator estimates the peak value by performing neural network learning using the coordinate-matched first ground reaction force value, the coordinate-matched foot pressure data, and the human body proportional mass parameter value.

13. A method of measuring a ground reaction force against a subject to be measured by a ground reaction force measuring apparatus, the method comprising:
measuring inertial data for the subject;
calculating a first ground reaction force value by using a human body proportional mass parameter value for the subject and the inertial data;
sensing a foot pressure for the subject;
calculating foot pressure data by using the foot pressure; and
calculating a second ground reaction force value by using the foot pressure data and the first ground reaction force value.

14. The method of claim 13, further comprising
sensing vision data for the subject,
wherein the calculating the first ground reaction force value includes calculating the first ground reaction force value by using the inertial data, the human body proportional mass parameter value, and the vision data.

15. The method of claim 13, wherein
the inertial data includes linear acceleration and rotation acceleration.

16. The method of claim 13, wherein
the foot pressure data includes a foot pressure distribution and a foot pressure center.

17. The method of claim 16, wherein
the calculating the foot pressure data includes calculating a walking period for the subject using the foot pressure distribution, and
the calculating a second ground reaction force value includes deleting a portion of the first ground reaction force value for a foot that is not in contact with a ground by using the walking period.

18. The method of claim 13, wherein
the calculating the second ground reaction force value includes:
calculating a weight movement ratio between both feet of the subject by using the foot pressure distribution or the foot pressure center; and
calculating the second ground reaction force value by reflecting the weight movement ratio to the first ground reaction force value.

19. A method of measuring a ground reaction force against a subject to be measured by a ground reaction force measuring apparatus, the method comprising:
measuring inertial data for the subject;
calculating a first ground reaction force value by using a human body proportional mass parameter value for the subject and the inertial data;
sensing a foot pressure for the subject;
calculating a walking period for the subject by using the foot pressure; and
calculating a final ground reaction force value by using the walking period and the first ground reaction force value.

20. The method of claim 19, further comprising
sensing vision data for the subject,
wherein the calculating the first ground reaction force value includes calculating the first ground reaction force value by using the inertial data, the human body proportional mass parameter value, and the vision data.
